(19) United States
(12) Patent Application Publication
(10) Pub. No.: US 2026/0345470 A1
(43) Pub. Date: Nov. 5, 2026

(54) COMPOSITION FOR PROMOTING HAIR GROWTH OR PREVENTING HAIR LOSS COMPRISING EXTRACTS OF PLATYCLADUS ORIENTALIS, LYCIUM CHINENSE MILL AND PIMPINELLA KOREANA

(71) Applicant: Stolive Co., Ltd, Chuncheon-si (KR)

(72) Inventor: Jun Kyoung KIM, Seoul (KR)

(73) Assignee: Stolive Co., Ltd, Chuncheon-si (KR)

(21) Appl. No.: 19/573,518

(22) Filed: Mar. 20, 2026

(30) Foreign Application Priority Data
Apr. 30, 2025 (KR) 10-2025-0056807

(51) Int. Cl. A61K 8/9761; A61K 8/49; A61K 8/9789; A61Q 7/00

(52) U.S. Cl. CPC A61K 8/9761; A61K 8/492; A61K 8/9789; A61Q 7/00; A61K 2800/10; A61K 2800/591

(57) ABSTRACT

The present disclosure relates to a composition for promoting hair growth or preventing hair loss, comprising extracts of Platycladus orientalis, Lycium chinense mill, and Pimpinella koreana. The composition exhibits effects of activating hair root cells and promoting hair formation and growth and thus may be usefully used for promoting hair growth, preventing hair loss, or treating hair loss.

### CROSS-REFERENCE TO RELATED APPLICATIONS

This application claims the priority of Korean Patent Application No. 10-2025-0056807 filed on Apr. 30, 2025, in the Korean Intellectual Property Office, the disclosure of which is incorporated herein by reference.

### BACKGROUND

### Field

The present disclosure relates to a composition for promoting hair growth or preventing hair loss including extracts of Platycladus orientalis, Lycium chinense mill, and Pimpinella koreana.

### Description of the Related Art

Hair develops when hair follicles, which are appendages of the skin, begin to form, and the hair follicles are small organs that exist on the body surface of mammals and repeat changes in a regular cycle. The hair follicle cycle is divided into anagen, catagen, and telogen. In the anagen, hair grows as the hair matrix cells and melanin-forming cells of the hair bulbs proliferate, in the catagen, the hair growth ends, the epithelial membrane contracts, and blood supply stops, and in the telogen, the hair is gradually pushed up to the hair follicle area to be lost.

The duration of the hair cycle varies depending on a body part, and hairs have a unique cycle including the catagen and the telogen after the anagen of approximately 26 years. However, the number of hair follicles in the anagen decreases with age, and hair loss occurs when the number of hairs is more than the average of 50 to 150 hairs per day that are lost naturally.

Hair loss refers to the absence of hairs in areas where hairs should normally exist, and not only has a cosmetic issue, but also affects important physiological functions such as UV protection and hair protection. The hair loss is broadly divided into scarring hair loss and non-scarring hair loss, and the scarring hair loss causes a serious problem in which hairs are not formed again due to permanent damage to the hair follicles.

Currently, therapeutic agents approved by the U.S. Food and Drug Administration (FDA) are Minoxidil as a topical application agent and Finasteride as an oral application agent. Minoxidil may prevent hair loss by expanding the capillaries in the scalp, but is inconvenient to be applied twice a day for a long time, and may be accompanied by side effects such as itching or irritation. Finasteride has a mechanism of suppressing hair loss by inhibiting the production of 5α-dihydrotestosterone (DHT) as a male hormone, but when stopping, the effect thereof is quickly lost and serious side effects such as decreased sexual function and risk of birth defects in women have been reported.

Therefore, the development of a novel composition capable of overcoming the limitations of conventional therapeutic agents is required to fundamentally solve hair loss. In particular, a need for developing therapeutic agents that promote hair growth by strengthening not only the hairs but also the hair roots and utilize safe, natural ingredients is increasingly emphasized.

Accordingly, the present inventors confirmed that a complex of extracts of Platycladus orientalis, Lycium chinense mill, and Pimpinella koreana induced the activity of hair root cells, thereby promoting hair formation and growth, and then completed the present disclosure.

### PRIOR ARTS

### Patent Documents

(Patent Document 1) Korean Patent Registration No. 10-2764550
(Patent Document 2) Korean Patent Publication No. 10-2024-0140872

### SUMMARY

An object of the present disclosure is to provide a pharmaceutical composition for promoting hair growth or preventing hair loss, including extracts of Platycladus orientalis, Lycium chinense mill, and Pimpinella koreana.

Further, another object of the present disclosure is to provide a cosmetic composition for promoting hair growth or preventing hair loss, including extracts of Platycladus orientalis, Lycium chinense mill, and Pimpinella koreana.

Further, yet another object of the present disclosure is to provide a food composition for promoting hair growth or preventing hair loss, including extracts of Platycladus orientalis, Lycium chinense mill, and Pimpinella koreana.

Further, still another object of the present disclosure is to provide a method for promoting hair growth, including administering or applying to a subject a composition including extracts of Platycladus orientalis, Lycium chinense mill, and
Pimpinella koreana.

In order to achieve the object, an aspect of the present disclosure provides a pharmaceutical composition for promoting hair growth or preventing hair loss, including extracts of

Platycladus orientalis, Lycium chinense mill, and Pimpinella koreana.

Further, another aspect of the present disclosure provides a cosmetic composition for promoting hair growth or preventing hair loss, including extracts of Platycladus orientalis, Lycium chinense mill, and Pimpinella koreana.

Further, yet another aspect of the present disclosure provides a food composition for promoting hair growth or preventing hair loss, including extracts of Platycladus orientalis, Lycium chinense mill, and Pimpinella koreana.

The extracts of Platycladus orientalis, Lycium chinense mill, and Pimpinella koreana may be extracted with a solvent selected from the group consisting of water, alcohols having 1 to 4 carbon atoms, or a mixed solvent thereof.

The extracts of Platycladus orientalis, Lycium chinense mill, and Pimpinella koreana may be mixed in a weight ratio of 0.5 to 3:0.5 to 3:0.5 to 3.

The extract of Platycladus orientalis may be a Platycladus orientalis leaf extract, the extract of Lycium chinense mill may be a Lycium chinense mill fruit extract, and the extract of Pimpinella koreana may be a Pimpinella koreana whole plant extract.

The composition may contain pheophorbide A.

The composition may activate hair root cells, and the activation of the hair root cells may promote proliferation of dermal papilla cells, germinal matrixes, or outer root sheath cells.

The composition may promote hair formation and hair growth.

The composition may increase the expression of IGF-1, VEGFA or Wnt3a.

Further, yet another aspect of the present disclosure provides a method for promoting hair growth or hair restoration including administering or applying a composition including extracts of Platycladus orientalis, Lycium chinense mill, and Pimpinella koreana to a subject other than humans.

Further, still another aspect of the present disclosure provides a method for activating hair root cells by treating hair follicle organoids in vitro with a composition including extracts of Platycladus orientalis, Lycium chinense mill, and Pimpinella koreana.

According to the present disclosure, the composition including the extracts of Platycladus orientalis, Lycium chinense mill, and Pimpinella koreana exhibits effects of activating hair root cells and promoting hair formation and growth, and thus can be usefully used for promoting hair growth, preventing hair loss, or treating hair loss.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 shows results of component analysis of a complex of extracts of Platycladus orientalis, Lycium chinense mill, and Pimpinella koreana;

FIG. 2 shows microscopic images and measurement criteria in hair follicle organoids treated with a complex of extracts of Platycladus orientalis, Lycium chinense mill, and Pimpinella koreana;

FIG. 3 shows microscopic photographs for each group in hair follicle organoids treated with a complex of extracts of Platycladus orientalis, Lycium chinense mill, and Pimpinella koreana;

FIG. 4 shows results of measuring hair follicle length, average hair length, and ratio of (number of hairs)/(total number of hair follicles) on day 20 for each group in hair follicle organoids treated with a complex of extracts of Platycladus orientalis, Lycium chinense mill, and Pimpinella koreana;

FIG. 5 shows H&E staining results in hair follicle organoids treated with a complex of extracts of Platycladus orientalis, Lycium chinense mill, and Pimpinella koreana;

FIG. 6 shows immunofluorescence staining results in hair follicle organoids treated with a complex of extracts of Platycladus orientalis, Lycium chinense mill, and Pimpinella koreana;

FIG. 7 shows results of measuring a ratio of Ki67-expressing cells per cell in a hair follicle matrix area in hair follicle organoids treated with a complex of extracts Platycladus orientalis, Lycium chinense mill, and Pimpinella koreana; and

FIG. 8 shows results of measuring the expression levels of IGF-1, VEGFα, and Wnt3α genes for each group in hair follicle organoids treated with a complex of extracts of Platycladus orientalis, Lycium chinense mill, and Pimpinella koreana.

### DETAILED DESCRIPTION OF THE EMBODIMENT

Hereinafter, aspects and exemplary embodiments of the present disclosure will be described in detail so as to be easily implemented by those skilled in the art, with reference to the accompanying drawings. However, the present disclosure may be implemented in various forms and is not limited to aspects and exemplary embodiments to be described herein.

Throughout the present specification, when a certain part “comprises” a certain component, unless otherwise disclosed to the contrary, it is meant that the part may further comprise another component without excluding another component.

The present disclosure provides a pharmaceutical composition for promoting hair growth or preventing hair loss, including extracts of Platycladus orientalis, Lycium chinense mill, and Pimpinella koreana.

The extract of Platycladus orientalis may be extracts of Platycladus orientalis, Thuja orientalis, etc., and preferably an extract of Platycladus orientalis, but is not limited thereto.

The extract of Platycladus orientalis may be extracts of leaves, fruits, bark, etc. of Platycladus orientalis, and preferably a Platycladus orientalis leaf extract, but is not limited thereto.

The extract of Lycium chinense mill may be extracts of Lycium chinense, Lycium barbarum, etc., and preferably an extract of Lycium chinense, but is not limited thereto.

The extract of Lycium chinense mill may be extracts of leaves, fruits, bark, etc. of Lycium chinense, and preferably an extract of fruits of Lycium chinense, but is not limited thereto.

The extract of Pimpinella koreana may be an extract of Pimpinella koreana or Ground Elder, and the name of Pimpinella koreana has room for confusion with Oenanthe javanica, which is an edible plant in Asia, but is a completely different plant.

The extract of Pimpinella koreana may be extracts of the aerial part, underground part, whole plant, etc. of Pimpinella koreana, and preferably an extract of the whole plant of Pimpinella koreana, but is not limited thereto.

The extracts of Platycladus orientalis, Lycium chinense mill, and Pimpinella koreana may be extracted with a solvent selected from the group consisting of water, alcohols having 1 to 4 carbon atoms, or a mixed solvent thereof, and preferably water, but are not limited thereto.

The composition may include a mixture (complex) mixed with the extracts of Platycladus orientalis, Lycium chinense mill, and Pimpinella koreana or an extract of a mixture of Platycladus orientalis, Lycium chinense mill, and Pimpinella koreana, but is not limited thereto.

The extracts of Platycladus orientalis, Lycium chinense mill, and Pimpinella koreana may be mixed in a weight ratio of 0.1 to 10:0.1 to 10:0.1 to 10, preferably in a weight ratio of 0.5 to 3:0.5 to 3:0.5 to 3, and more preferably in a weight ratio of 1:1:1, but is not limited thereto.

The composition may contain pheophorbide A, and the pheophorbide A may act as an active ingredient, but is not limited thereto.

The composition may contain pheophorbide A in the content of 0.0001 to 0.001 μg/mg, preferably 0.0001 to 0.0005 μg/mg, and more preferably 0.00016 to 0.00039 μg/mg, but is not limited thereto.

The composition may induce promotion of hair growth or prevention of hair loss by activating hair root cells, and may have different mechanisms of action from minoxidil, which is known to induce the prevention of hair loss or promotion of weak hair growth by expanding scalp blood vessels, and finasteride, which is known to prevent hair loss by inhibiting a hormone (DHT) involved in hair loss, but is not limited thereto.

The hair root cells may include dermal papilla cells, germinal matrixes, and outer root sheath cells, and the activation of the hair root cells may promote the proliferation of dermal papilla cells, germinal matrixes, or outer root sheath cells, but are not limited thereto.

The dermal papilla cells are fibroblasts derived from the mesoderm and are known to affect the hair cycle through the secretion of proteins such as insulin-like growth factor-1 (IGF-1), hepatocyte growth factor (HGF), and fibroblast growth factor-7 (FGF-7).

The germinal matrix is a region of the hair follicle that plays a key role in hair formation and growth, and the cells in the germinal matrix divide very quickly and form the components of hair through a keratinization process. In particular, the germinal matrix is known to form hair fibers as a main structure of hairs, thereby contributing to the continuous growth of hairs.

The composition may promote hair formation and may promote hair growth, but is not limited thereto.

The composition may promote the growth of hair follicles and may activate the proliferation of hair follicle cells, but is not limited thereto.

The composition may increase the expression of IGF-1, VEGFA or Wnt3a, and preferably in hair root cells, but is not limited thereto.

The insulin-like growth factor 1 (IGF-1) plays an important role in promoting hair follicle growth and proliferation, and is known to protect hair follicle cells by inhibiting self-death, prevent hair loss, and promote hair growth by activating a Wnt/β-catenin signaling pathway.

The vascular endothelial growth factor A (VEGFA) is a vascular endothelial growth factor and is known to improve blood circulation by growing the vascular endothelium, extend the anagen of hair follicles, promote cell survival, and inhibit cell death, thereby enhancing hair growth and differentiation of hair root cells.

The Wnt3a activates a Wnt/β-catenin signaling pathway to induce β-catenin stabilization, stimulates hair follicle stem cells, promote hair follicle regeneration and growth, and inhibits rapid transition of hair follicles to catagen to maintain the hair follicle function for a longer period of time, thereby affecting hair growth.

The activity of the Wnt/β-catenin signaling pathway is known to play a role in skin formation and hair follicle development, and is known to inhibit hair growth in mice in which a Wnt gene is knocked down. Accordingly, substances that promote the activation of the Wnt/β-catenin signaling pathway present in hair follicle stem cells may promote hair growth.

The composition of the present disclosure may increase the proliferation of hair root cells such as dermal papilla cells, and significantly increase the expression of hair growth-related factors and cytokines and the activation of Wnt/β-catenin signaling proteins, but is not limited thereto.

The pharmaceutical composition of the present disclosure may be formulated into powders, granules, tablets, coated tablets, pills, sugar-coated tablets, capsules, liquids, suspensions, gels, syrups, slurry agents, suppositories, emulsions, pastes, ointments, creams, lotions, powders, sprays or suspensions. The pharmaceutical composition may further include excipients, such as fillers, extenders, binders, wetting agents, disintegrants, surfactants, and diluents, which are commonly for the formulation, and in addition to simple excipients, lubricants such as magnesium stearate and talc may also be used, but are not limited thereto.

The pharmaceutical composition may be administered parenterally or orally depending on an intended method, and the dose range varies depending on the weight, age, sex, health condition of a patient, diet, administration time, administration method, excretion rate, and severity of a disease. In addition, a therapeutically effective amount of the composition may vary depending on an administration method, a target site, and a patient's condition, and when used in the human body, the dose needs to be determined as an appropriate amount in consideration of both safety and effectiveness.

The pharmaceutical composition may further include an adjuvant in addition to the active ingredients, and any adjuvant known in the art may be used as the adjuvant without limitation.

Further, the present disclosure provides a cosmetic composition for promoting hair growth or preventing hair loss, including extracts of Platycladus orientalis, Lycium chinense mill, and Pimpinella koreana.

The cosmetic composition of the present disclosure may be prepared even in any formulation commonly prepared in the art, and may be formulated into, for example, a solution, a suspension, an emulsion, a paste, a gel, a cream, a lotion, a powder, a soap, a surfactant-containing cleansing, oil, a powder foundation, an emulsion foundation, a wax foundation, a spray, and the like, but is not limited thereto. More specifically, the cosmetic composition of the present disclosure may be prepared in formulations of a skin toner (skin), a nourishing toner (milk lotion), a nourishing cream, a massage cream, an essence, an eye cream, a cleansing cream, a cleansing foam, cleansing water, packs, a spray or powder. In addition to the active ingredients, the cosmetic composition of each formulation may be mixed with ingredients commonly used in the art, which are appropriately selected by those skilled in the art without difficulty according to a formulation or purpose of use.

The cosmetic composition may be used as a scalp or hair composition, etc., and may be formulated into formulations used for both the scalp and hair, that is, scalp or hair, and scalp and hair combined products, such as shampoo, rinse, scalp treatment, hair treatment, scalp and hair combined treatment, hair treatment for preventing hair loss, treatment for damaged hair, leave-in conditioner, wash-off conditioner, or hair essence, but is not limited thereto.

The cosmetic composition may contain adjuvants commonly used in the field of cosmetology or dermatology, such as fatty substances, organic solvents, solubilizers, thickeners, gelling agents, softeners, antioxidants, suspending agents, stabilizers, foaming agents, fragrances, surfactants, water, ionic or nonionic emulsifiers, fillers, metal ions sequestering agents, chelating agents, preservatives, vitamins, blocking agents, humectants, essential oils, dyes, pigments, hydrophilic or lipophilic actives, lipid vesicles or any other ingredients commonly used in cosmetics.

Further, the present disclosure provides a food composition for promoting hair growth or preventing hair loss, including extracts of Platycladus orientalis, Lycium chinense mill, and Pimpinella koreana.

The food composition of the present disclosure may be health functional foods, dairy products, fermented products, or food additives. The health functional food refers to food produced and processed using raw materials or ingredients with functionality, which are useful for the human body, and the “functionality” means intake for adjusting nutrients for the structures and functions of the human body or obtaining a useful effect on health applications such as physiological actions.

The food composition includes formulations such as pills, powders, granules, infusions, tablets, capsules, liquids, pastes, gels, or jellies, and the food composition of each formulation may be mixed with ingredients commonly used in the art, which are appropriately selected by those skilled in the art without difficulty according to a formulation or purpose of use, in addition to the active ingredients.

Further, the present disclosure provides a method for promoting hair growth or hair restoration, including administering to a subject other than humans or applying or spraying on the skin a composition including extracts of Platycladus orientalis, Lycium chinense mill, and Pimpinella koreana.

Further, the present disclosure provides a method for activating hair root cells by treating hair follicles or skin organoids, or hair root cells in vitro with a composition including extracts of Platycladus orientalis, Lycium chinense mill, and Pimpinella koreana.

In the present disclosure, “hair growth” means hair growing in hair follicles. The hair includes hair roots and hair follicles of the head, hairs, eyelashes and eyebrows, beard, armpits, pubic hair, and all parts of the entire body with hair roots and hair follicles.

In the present disclosure, “promotion of hair growth” means stimulating hair growth, ultimately increasing the proportion of hairs in the anagen among all hairs. Therefore, the term “promotion of hair growth” may have the same meaning as “improvement of hair growth”, by suppressing hair loss caused by a decrease in the proportion of hairs in the anagen.

In the present disclosure, “hair restoration” means raising hair, and broadly growing the roots, circumference, length, strength, etc. of hair by increasing and strengthening the thickness of hair.

In the present disclosure, “hair loss” includes not only hair falling out from the scalp, but also a decrease in hair density or a decrease in hair thickness.

In the present disclosure, “improvement of hair loss” refers to the action of preventing hair loss by weakening the catagen of hair follicles and inducing the anagen. Therefore, the term “improvement of hair loss” may have the same meaning as “alleviation of hair loss”, “prevention of hair loss”, and “treatment of hair loss” by inhibiting hair loss caused by a decrease in the proportion of hairs in the anagen.

As used in the present disclosure, the term “extract” refers to an active ingredient isolated from a natural product, that is, a substance showing a desired activity. The extract may be obtained through an extraction process using water, an organic solvent, or a mixed solvent thereof, and includes dry powders of the extract or all forms formulated using the dry powders. In addition, the extract includes fractions of the extract that has been subjected to the extraction process. The extraction method of the extract is not particularly limited, and may be extracted by, for example, stirring extraction, shaking extraction, hot water extraction, cold immersion extraction, reflux cooling extraction, or ultrasonic extraction. The extraction solvent may also be used with a polar solvent such as water and lower alcohols of C1-C4, a non-polar solvent such as hexane, chloroform, dichloromethane or ethyl acetate, or a mixture of two or more thereof.

As used in the present disclosure, the term “prevention” means all actions of inhibiting or delaying gastrointestinal diseases, etc. by administering the composition according to the present disclosure. The “treatment” means all actions of improving or beneficially changing the symptoms of a suspected or developed subject for gastrointestinal diseases by administering the composition. In addition, the “improvement” means all actions that at least reduce parameters associated with conditions to be treated, for example, the degree of symptoms by administering the composition including the extracts of the present disclosure.

In the present disclosure, used to indicate the concentration of a specific material is solid/solid (w/w) %, solid/liquid (w/v) %, and liquid/liquid (v/v) %, unless otherwise stated.

The data of the present disclosure were analyzed using Excel and Prism, and were expressed as mean and standard errors. Through statistical processing, it was determined to have statistical significance when a P value calculated using Tukey's test was less than 0.05 (P value; *<0.05, **<0.01, ***<0.001).

Hereinafter, the present disclosure will be described in more detail with reference to the following Examples, but the following Examples are only for illustrative purposes and are not intended to limit the scope of the present disclosure.

### [Preparation Example] Preparation of Extracts

### 1. Preparation of Extract of Platycladus orientalis

The dried leaves of Platycladus orientalis (L.) Franco (Makecorp Co., Ltd.) were crushed and prepared. The crushed leaves (3 g) of Platycladus orientalis (L.) Franco were added with 100 ml of purified water, heated in a heating stirrer (Corning PC-420) without boiling (at approximately 80 °C), evaporated until the total volume became approximately 30 ml, and then centrifuged at 3000 rpm for 20 minutes to obtain 30 ml of a first extract. The precipitate was added with 50 ml of purified water, heated in the same manner, evaporated until the total volume became 20 ml, and then centrifuged at 3000 rpm for 20 minutes to obtain 20 ml of a second extract. The first extract (30 ml) and the second extract (20 ml) were mixed, heated in the same manner, and then concentrated until the total volume became approximately 3 ml to obtain an extract of Platycladus orientalis.

### 2. Preparation of Extract of Lycium chinense Mill

An extract of Lycium chinense mill was obtained in the same manner as in Preparation Example 1-1, except that the dried fruit of Lycium chinense Mill. (Makecorp Co., Ltd.) was used.

### 3. Preparation of Extract of Pimpinella koreana

An extract of Pimpinella koreana was obtained in the same manner as in Preparation Example 1-1, except that the dried whole plant of Pimpinella koreana (Makecorp Co., Ltd.) was used.

### [Example 1] Preparation of Complex of Extracts of Platycladus orientalis, Lycium Chinense Mill, and Pimpinella koreana

The extracts of Platycladus orientalis, Lycium chinense mill, and Pimpinella koreana prepared in Preparation Examples 1 to 3 above were mixed in a weight ratio of 1:1:1, and then added with distilled water to make a concentration of 5 wt % to prepare a complex of the extracts, and then the complex was used in subsequent experiments.

### [Example 2] Preparation of Human Hair Follicle Organoids and Sample Treatment

### 2-1. Preparation of Skin Organoids

Skin organoids were prepared based on a method disclosed in Korean Patent Registration No. 10-2764550. Induced pluripotent stem cell lines (iPSCs; CMC3) derived from healthy subjects were incubated on Vitronectin (ThermoFisher)-coated culture dishes without feeder cells in an Essential 8 (Gibco) culture medium supplemented with Y-27632 (10 μM). The culture medium was replaced daily, and subcultured using ReLeSR™ (Stem Cell Technology) at about 5-day intervals. Skin organoids were incubated with reference to the literature (Lee, J. et al. Hair-bearing human skin generated entirely from pluripotent stem cells. Nature 582, 399-404 (2020)) that disclosed an existing skin organoid differentiation protocol using the induced pluripotent stem cell lines. Induced pluripotent stem cell colonies were separated into small cell cluster units using ReLeSR™ (Stem Cell Technology), and then separated into single cells using Accutase (ThermoFisher). For embryoid body formation, 1000 single cells were dispensed into respective wells of a U-bottom low-attachment 96-well plate (Corning). The embryoid body culture medium was used with Essential 8 (Gibco) supplemented with Y-27632 (20 UM). The embryoid bodies were incubated for 2 days until growing to a size of 300 to 500 μm. On day 2 of differentiation, in order to induce non-neural ectoderm differentiation, the formed embryoid bodies transferred to a culture medium containing 2% Matrigel were (Corning), 10 μM SB431542 (Tocris), 4 ng/ml FGF2 (PeproTech), and 15 ng/ml BMP4 (PeproTech). From day 6, the culture medium was added with 200 ng/ml of FGF2 and 50 μg/ml of LDN193189 (Tocris) to induce cranial neural crest cells. On day 14, the organoids were transferred to a 6-well plate (low-attachment 6-well plate; Corning) containing 3 ml of a skin organoid maturation medium and skin organoids were incubated on an Orbital shaker (65 rpm; Thermo Fisher). The culture medium was replaced every three days. The skin organoid maturation medium contained Advanced DMEM/F12 (Gibco) and Neurobasal (Gibco) media in a 1:1 ratio, supplemented with 1× GlutaMax™ (Gibco), 0.5× B-27 minus vitamin 1 A (Gibco), 0.5×N2 (Gibco), a supplement, 0.1 mM 2-mercaptoethanol (Gibco), and 100 μg/ml Normocin (Invivogen).

### 2-2. Preparation of Hair Follicle Strips

Hair follicle strips were prepared based on a method disclosed in Korean Patent Publication No. 10-2024-0140872. The skin organoids were transferred to a tissue culture plate and cut using a Spring Micro-Scissor, and then a target tissue was separated from an epidermal layer and a dermal layer using forceps. The skin organoids were highly embedded into the collagen fibers of hair follicles and skin layers (epidermal and dermal layers) and tended to adhere tightly and thus were carefully removed with microneedles. Hair follicles at the hair germ stage or higher were isolated, and the isolated hair follicles were washed with a culture medium to avoid contamination from other cells, and then incubated to be exposed to air on transwell culture inserts 3D-coated with collagen.

### 2-3. Sample Treatment

After stabilization in a 37 °C incubator for 7 days, 20 μl of each sample was directly applied to the hair follicles on the transwell culture inserts once every two days for a total of 20 days. As the sample, a complex of the extracts of Platycladus orientalis, Lycium chinense mill, and Pimpinella koreana prepared in Example 1 was used, and a mixed solvent of 5% butylene glycol and 2% 1,2-hexanediol was used as a negative control (NC), and minoxidil was used as a positive control (PC).

### [Experimental Example 1] Component Analysis of Complex of Extracts of Platycladus orientalis, Lycium Chinense Mill, and Pimpinella koreana

Ingredients contained in the complex of extracts of Platycladus orientalis, Lycium chinense mill, and Pimpinella koreana prepared in Example 1 were analyzed. LC/MS analysis was performed using UPLC-Q-TOF MS (Xevo™ TQ-S micro, Waters, Milford, MA, USA), and the analysis conditions were as shown in Table 1 below.

TABLE 1

| | |
|---|---|
| inject volume | 1 μL |
| Column | Acquity UPLC BEH C18 column (2.1 mm × 100 mm, |
| | 1.7 μm; Waters) |
| Solvent A | water with 0.1% formic acid (FA) |
| Solvent B | ACN with 0.1% FA |
| Flow rate | 0.35 mL/min |

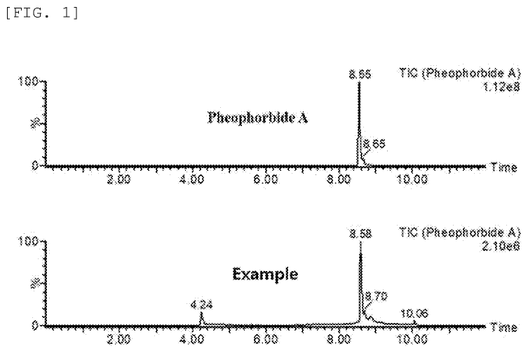

As a result, as shown in Table 2 and FIG. 1 below, it was found that the complex of Example 1 contained pheophorbide A, and it was confirmed that the content of pheophorbide A was 0.00016 to 0.00039 μg/mg (average 0.00026 μg/mg).

TABLE 2

| Sample | Content (μg/mg) |
|---|---|
| Complex 1 of Example 1 | 0.00039 |
| Complex 2 of Example 1 | 0.00023 |
| Complex 3 of Example 1 | 0.00016 |

### [Experimental Example 2] Evaluation of Hair Follicle Growth, Hair Formation, and Hair Length Growth

The hair follicle organoids of Example 2 above were photographed using a microscope (EVOS XL Core) on days 0 and 20 of sample treatment. The hair follicle growth of each group was compared as (the length (D20) of hair follicle measured on day 20)/(the length (DO) of hair follicle measured on day 0), and the average value was calculated for each group. In addition, the lengths of the hair follicles (hairs) of each group were measured using the ImageJ program, and the growth and presence of hair in each hair follicle were measured for each group, and the average values were calculated.

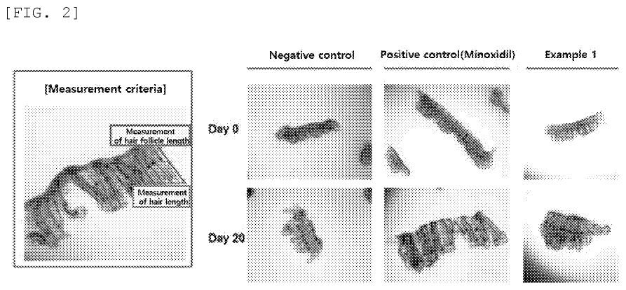

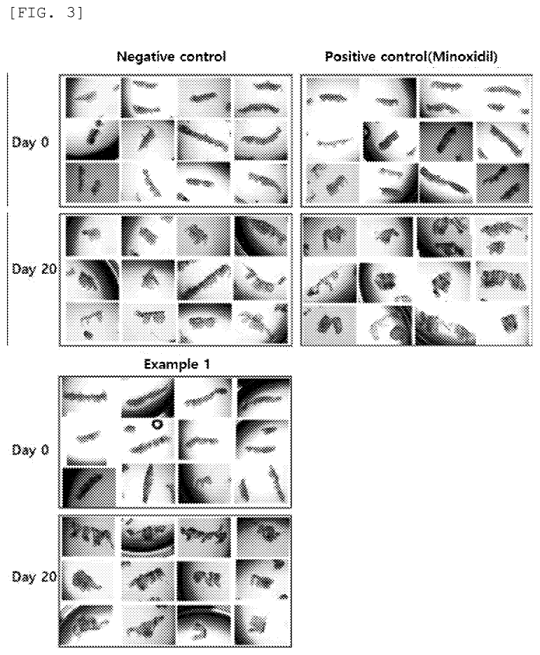

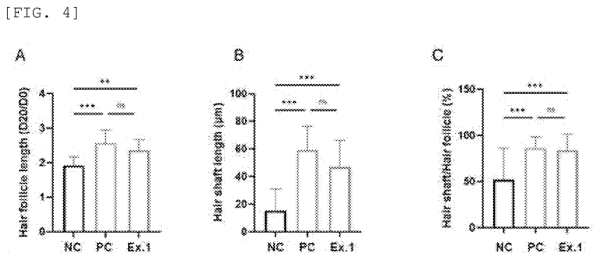

As a result, as shown in FIGS. 2 to 4, as compared to day 0 of sample treatment, the hair follicle length was 1.92 times in a negative control group, but increased to 2.57 times in a positive control group, and 2.36 times in an Example 1 treatment group, and thus it was confirmed that the hair follicles grew significantly longer. Hair formation in the hair follicles was only 52.31% in the negative control group (hair formation in 87 out of 167 hair follicles), but was 86.43% in the positive control group (hair formation in 159 out of 184 hair follicles), and 84.11% in the Example 1 treatment group (hair formation in 152 out of 181 hair follicles), and thus it was confirmed that the hair was significantly formed. Even in the hair length, the negative control group was 15.40 μm, the positive control group was 59.21 μm, and the Example 1 treatment group was 49.45 μm, and thus it was confirmed that the hair length was significantly increased.

Therefore, it was confirmed that the complex of the extracts of Platycladus orientalis, Lycium chinense mill, and Pimpinella koreana of the present disclosure induced hair formation and growth.

### [Experimental Example 3] Histopathological Evaluation of Hair Follicle Organoids

Hair follicle organoids on day 20 of sample treatment were fixed in a 4% PFA solution and dehydrated in 30% sucrose. The organoids were embedded in a gelatin/sucrose solution, and then sectioned into blocks using a cryostat microtome. Hematoxylin & Eosin staining was performed and the thickness, length, and degree of hair formation of the hair follicles were evaluated under a microscope (X100).

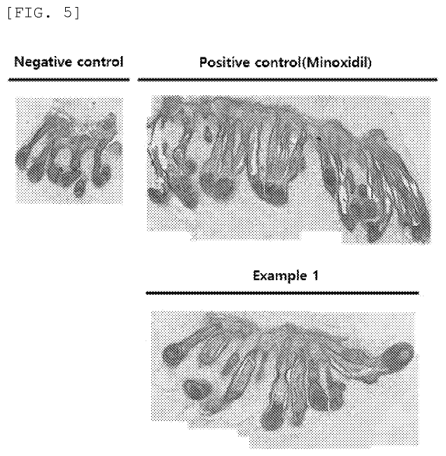

As a result, as shown in FIG. 5, in the negative control group, the hair follicles grew thin and short, whereas in both the positive control group and the Example 1 treatment group, the hair bulbs grew thicker, the length of the hair follicles grew longer, and a lot of hair was formed.

Therefore, it was confirmed that the complex of the extracts of Platycladus orientalis, Lycium chinense mill, and Pimpinella koreana of the present disclosure induced hair formation and growth.

### [Experimental Example 4] Immunofluorescence Staining Evaluation of Hair Follicle Organoids

Hair follicle organoids on day 20 of sample treatment were fixed in a 4% PFA solution and dehydrated in 30% sucrose. The organoids were embedded in a gelatin/sucrose solution, and then sectioned into blocks using a cryostat microtome. To expose antigens in the fixed organoid sections, the antigens were recovered at 95 °C using a sodium citrate solution and then the tissues were washed with PBS. To increase cell membrane permeability, the tissues were permeabilized with 0.2% Triton X-100, washed, and then non-specific antibody binding was blocked using 5% normal goat serum for 1 hour. Then, the tissues were stained with anti-Ki67 antibody (BD pharmigen, Cat #550609) and anti-KRT 5 antibody (Abcam, Cat #ab52635) at 4 °C for 24 hours. After secondary antibody treatment, the tissues were subjected to nuclear staining and confirmed using a confocal scanning microscope, and a ratio of Ki67-expressing cells per hair follicle matrix was counted. The Ki67 was a major marker indicating cell proliferation, and the increased expression of Ki67 in hair follicle cells indicated that cell proliferation activity within the hair follicles was promoted.

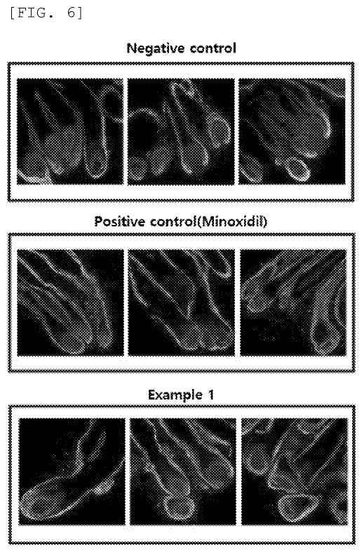

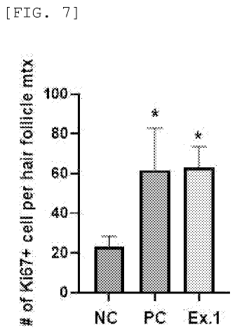

As shown in FIGS. 6 and 7, as a result of immunofluorescence staining using anti-ki67 antibody and anti-KRT5 antibody staining outer root sheath cells, the Ki67 expression within the hair follicle matrix structure was significantly increased in the Example 1 treatment group compared to the negative control group. This indicated that the cell division rate within the hair follicles was accelerated in the Example 1 treatment group, which suggested that hair formation was actively achieved. It was meant that the activation of cell proliferation within the hair follicles played an important role in hair growth, and the complex of Example 1 had a positive effect in inducing the activity of hair root cells, thereby stimulating the hair growth cycle and promoting hair growth.

Therefore, it was confirmed that the complex of the extracts of Platycladus orientalis, Lycium chinense mill, and Pimpinella koreana of the present disclosure induced the activation of hair root cells to proliferate hair root cells, thereby promoting the hair growth.

### [Experimental Example 5] Evaluation of Gene Expression Levels in Hair Follicle Organoids

To confirm the expression levels of IGF-1, VEGFA, and Wnt3a, as factors affecting hair growth, from hair follicle organoids on day 20 of sample treatment, the mRNA expression of each gene was analyzed using polymerase chain reaction analysis (Reverse transcription-PCR analysis). The hair follicle organoids were washed with PBS, added with 1 ml of TRIzol (Invitrogen, Carlsbad, CA, USA), added with beads, and pulverized with a TissueLyser to extract total RNA. qRT-PCR analysis was performed using SYBR Green PCR Mix (Applied Biosystems, USA) and 7500 Real-Time PCR System (Applied Biosystems). The relative mRNA expression of each target gene was quantified using a 2 (AA threshold cycle) (2-ΔΔCT) method and normalized according to glyceraldehyde-3-phosphate dehydrogenase (GAPDH).

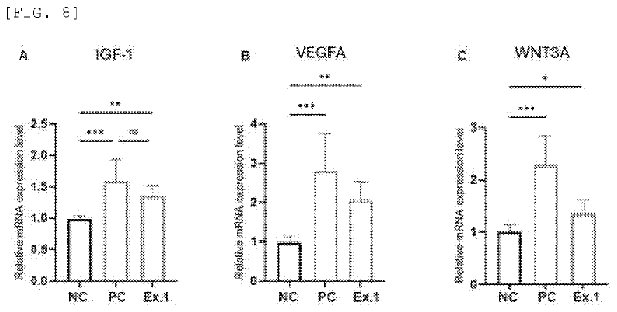

As a result, as shown in FIG. 8, it was confirmed that the expression of IGF-1, VEGFA, and Wnt3a was all increased in the Example 1 treatment group, which was significantly increased compared to the negative control group.

Therefore, it was confirmed that the complex of the extracts of Platycladus orientalis, Lycium chinense mill, and Pimpinella koreana of the present disclosure exhibited the effect of promoting hair growth.

### Preparation Example

The extracts according to the present disclosure may be formulated in various forms depending on a purpose. Hereinafter, some formulation methods containing the extracts according to the present disclosure as active ingredients will be exemplified, but the present disclosure is not limited thereto.

### 1. Tablets

100 mg of the complex of Example 1 of the present disclosure, 400 mg of lactose, 400 mg of corn starch, and 2 mg of magnesium stearate were mixed and then tableted according to a conventional tablet preparation method to prepare tablets.

### 2. Granules

50 mg of the complex of Example 1 of the present disclosure, 250 mg of anhydrous crystalline glucose, and 550 mg of starch were mixed, formed into granules using a fluidized bed granulator, and then filled into bags.

### 3. Capsules

100 mg of the complex of Example 1 of the present disclosure, 400 mg of lactose, 400 mg of corn starch, and 2 mg of magnesium stearate were mixed and then filled into gelatin capsules according to a conventional capsule preparation method to prepare capsules.

4. Drinks 50 mg of the complex of Example 1 of the present disclosure, 10 g of glucose, 0.6 g of citric acid, and 25 g of liquid oligosaccharide were mixed and then added with 300 ml of purified water to be filled into 200 ml of each bottle. The mixture was filled into bottles and sterilized at 130 °C for 4 to 5 seconds or at 80 °C for 10 to 30 seconds to prepare drinks.

### 5. Jellies

50 mg of the complex of Example 1 of the present disclosure, 36 mg of food gel, 6 mg of carrageenan, 10 mg of calcium gluconate, 6 mg of sodium citrate, and 6 g of oligosaccharide were mixed to prepare jellies according to a conventional jelly preparation method.

### 6. Skin Toner

A skin toner was prepared by a conventional method according to a composition shown in Table 3 below.

TABLE 3

| Ingredient | Content (wt %) |
|---|---|
| Complex of Example 1 | 0.2 |
| Glycerin | 3.0 |
| Butylene glycol | 2.0 |
| Propylene glycol | 2.0 |
| Carboxyvinyl polymer | 0.1 |
| Polyglyceryl-10 laurate | 0.2 |
| Polysorbate 80 | 0.4 |
| Ethanol | 10.0 |
| Sodium hyaluronate | 0.1 |
| Preservative, pigment, fragrance | Appropriate amount |
| Purified water | Remaining amount |

### 7. Nourishing Toner

A nourishing toner was prepared by a conventional method according to a composition shown in Table 4 below.

TABLE 4

| Ingredient | Content (wt %) |
|---|---|
| Complex of Example 1 | 1.0 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalene | 5.0 |
| Sorbitase sesquioleate | 1.5 |
| Jojoba oil | 0.5 |
| Cetearyl alcohol | 1.0 |
| Panthenol | 0.2 |
| Preservative, pigment, fragrance | Appropriate amount |
| Purified water | Remaining amount |

### 8. Massage Cream

A massage cream was prepared by a general method according to a composition shown in Table 5 below.

TABLE 5

| Ingredient | Content (wt %) |
|---|---|
| Complex of Example 1 | 2.0 |
| Glycerin | 8.0 |
| Butylene glycol | 4.0 |
| Macadamia seed oil | 45.0 |
| Beta-glucan | 7.0 |
| Carbomer | 0.1 |
| Caprylic/capric triglyceride | 3.0 |
| Beeswax | 4.0 |
| Cetearyl glucoside | 1.5 |
| Sorbitan sesquioleate | 0.9 |
| Shea butter | 3.0 |
| Paraffin | 1.5 |
| Preservative, pigment, fragrance | Appropriate amount |
| Purified water | Remaining amount |

### 9. Preparation of Hair Lotion

A hair lotion was prepared by a general method according to a composition shown in Table 6 below.

TABLE 6

| Ingredient | Content (wt %) |
|---|---|
| Complex of Example 1 | 2.0 |
| Purified water | Remaining amount |
| Glycerin | 3.0 |
| Butylene glycol | 3.0 |
| Jojoba oil | 7.0 |
| Beta-glucan | 7.0 |
| Carbomer | 0.1 |
| Edelweiss extract | 2.0 |
| Caprylic/capric triglyceride | 3.0 |
| Squalene | 5.0 |
| Cetearyl glucoside | 1.5 |
| Sorbitan stearate | 0.4 |
| Polyglyceryl-10 laurate | 1.2 |
| Preservative | Appropriate amount |
| Fragrance | Appropriate amount |
| Pigment | Appropriate amount |
| Panthenol | 0.1 |

### 10. Preparation of Shampoo Composition

A shampoo composition was prepared by a general method according to a composition shown in Table 7 below.

TABLE 7

| Ingredient | Content (wt %) |
|---|---|
| Polyquaternium-10 | 0.5 |
| Disodium laureth sulfosuccinate | 20 |
| Complex of Example 1 | 0.3 |
| Fragrance | 1.0 |
| Phenoxyethanol | 0.1 |
| Cetyl alcohol | 0.5 |
| Sodium chloride | 0.8 |
| Purified water | Remaining amount |

### 11. Preparation of Hair Treatment

A hair treatment was prepared by a general method according to a composition shown in Table 8 below.

TABLE 8

| Ingredient | Content (wt %) |
|---|---|
| Complex of Example 1 | 0.5 |
| Behentrimonium chloride | 2 |
| Glycerin | 0.5 |
| Fragrance | 0.5 |
| Preservative | 0.03 |
| Cetostearyl alcohol | 5 |
| Lactic acid | 1 |
| Distilled water | Remaining amount |

What is claimed is:

1. A method for promoting hair growth or preventing hair loss, comprising administering to a subject in need thereof extracts of Platycladus orientalis, Lycium chinense mill, and Pimpinella koreana.

2. The method of claim 1, wherein the extracts of Platycladus orientalis, Lycium chinense mill, and Pimpinella koreana are extracted with a solvent selected from the group consisting of water, alcohols having 1 to 4 carbon atoms, or a mixed solvent thereof.

3. The method of claim 1, wherein the extracts of Platycladus orientalis, Lycium chinense mill, and Pimpinella koreana are mixed in a weight ratio of 0.5 to 3:0.5 to 3:0.5 to 3.

4. The method of claim 1, wherein the extract of Platycladus orientalis is a Platycladus orientalis leaf extract, the extract of Lycium chinense mill is a Lycium chinense mill fruit extract, and the extract of Pimpinella koreana is a Pimpinella koreana whole plant extract.

5. The method of claim 1, wherein the extracts contain pheophorbide A.

6. The method of claim 1, wherein the extracts activate hair root cells.

7. The method of claim 6, wherein the activation of the hair root cells promotes proliferation of dermal papilla cells, germinal matrixes, or outer root sheath cells.

8. The method of claim 1, wherein the extracts promote hair formation.

9. The method of claim 1, wherein the extracts promote hair growth.

10. The method of claim 1, wherein the extracts increase the expression of IGF-1, VEGFA or Wnt3a.

11. A method for promoting hair growth or hair restoration, comprising administering or applying a composition including extracts of Platycladus orientalis, Lycium chinense mill, and Pimpinella koreana to a subject other than humans.
